(19) United States
(12) Patent Application Publication
(10) Pub. No.: US 2009/0060215 A1
(43) Pub. Date: Mar. 5, 2009

(54) Portable amplified stethoscope with recording capability

(76) Inventor: Luz M. Ocasio, Bronx, NY (US)

Correspondence Address: LAW OFFICE OF KYLE FLETCHER, P.O. Box 11735, CHARLOTTE, NC, 28220, US

(21) Appl. No.: 11/897,450

(22) Filed: Aug. 30, 2007

(51) Int. Cl. A61B 7/02

(52) U.S. Cl. 381/67

(57) ABSTRACT

The present invention relates to an improved design for a stethoscope. The invention is a stethoscope with a built-in amplifier and a memory stick to digitally record sounds as well as patient information. The present invention will be able to digitally record the sounds from the stethoscope through the use of an electronic amplification system and a built in memory stick. This system will also have a USB Port that allows the invention to communicate with a computer system. The medical practitioner will also be able to digitally hear the patient sounds with a set of head-phones or through the use of a Bluetooth system.

### CROSS REFERENCES TO RELATED APPLICATIONS

This application is a continuation-in-part to the Non-provisional Patent Application No.: 11/897,450, which was filed on Aug. 30, 2007.

### STATEMENT REGARDING FEDERALLY SPONSORED RESEARCH

Not Applicable

### REFERENCE TO APPENDIX

Not Applicable

### BACKGROUND OF THE INVENTION

A. Field of the Invention

The present invention relates to an improved design for a stethoscope. The invention is a stethoscope with a built-in amplifier and a memory stick to digitally record sounds as well as patient information.

In the past medical practitioners have not been able to accurately document the sounds coming from conventional stethoscopes. The present invention seeks to digitally record said sounds, be capable of connecting to a computer, and have Bluetooth capabilities.

B. Discussion of the Prior Art

The Simms Patent (U.S. Pat. No. 6,340,350) discloses an electronic stethoscope and belt-clipable housing comprising of a chest piece, an earpiece, and a casing for holding the chest piece and the earpiece. However, electronic stethoscope of the Simms Patent does not have the ability to record sound nor does the Simms Patient have a USB outlet to provide communication with a computer.

The Thomasson Patent (U.S. Pat. No. 5,557,681) discloses an electronic stethoscope which includes a microphone mounted in a resilient collar in a hand-held case. However, the electronic stethoscope of the Thomasson Patent does not have the ability to record sound nor does the Thomason Patient have a USB outlet to provide communication with a computer.

The Carman Patent Application Publication (U.S. Pub. No. 2001/0050992) discloses a two piece wireless electronic stethoscope designed to auscultate biological sounds in the course of medical practice. However, the electronic stethoscope of the Carman publication does not have the ability to record sound, does not include a USB outlet for communication with a computer, and is not Bluetooth capable.

The Orten Patent Application Publication (U.S. Pub. No. 2004/0223621) discloses a hand-held and hand-operated stethoscope sensor module with optional wireless headset. However, the electronic stethoscope of the Orten publication does not also provide the ability to record sound nor does device include a USB outlet for communication with a computer.

The Werblud Patent Application Publication (U.S. Pub. No. 2003/0002685) discloses an electric auscultation aid with the ability to wirelessly transmit detected signals to one or more remote devices. However, the electronic stethoscope of the Werblud publication does not have an ability to record sound nor does it include a USB outlet.

The Sommerfeld Design Patent et al. (U.S. Pat. No. Des. 477,874) discloses an ornamental design for an electronic stethoscope, which does not illustrate a USB port, or integrated recording means.

In light of the above discussed prior art, there is a need for an electronic stethoscope having a recording capability, a USB port for connectivity with a computer, and Bluetooth capability integrated into the device.

### BRIEF SUMMARY OF THE INVENTION

The present invention relates to an improved design for a stethoscope. The present invention comprises a housing, which includes a stethoscope, ON/OFF switch, volume control, integrated head phones set that is either wired or wireless, a USB port, built-in amplification means, and memory stick. The medical practitioner will be able to digitally hear the patient sounds with a set of head-phones or through the use of a Bluetooth system.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention:

In the drawings:

FIG. 1 illustrates an isometric view of the invention with the retractable wired head phones;

FIG. 2 illustrates a front view of the invention with the retractable wired head phones;

FIG. 3 illustrates a front view of the invention with the retractable wired head phones in an extended position;

FIG. 4 illustrates a rear view of the invention with the retractable wired head phones;

FIG. 5 illustrates a diagram of the electrical componentry of the invention with the retractable wired head phones;

FIG. 6 illustrates an isometric view of the invention in use with the wireless head phone set;

FIG. 7 illustrates an isometric view of the flip-up embodiment;

FIG. 8 illustrates a front view of the head piece that is used with the flip-up embodiment;

FIG. 9 illustrates a side view of the flip-up embodiment;

FIG. 10 illustrates a rear view of the flip-up embodiment; and

FIG. 11 illustrates an electrical diagram of the flip-up embodiment.

### DETAILED DESCRIPTION OF THE EMBODIMENT

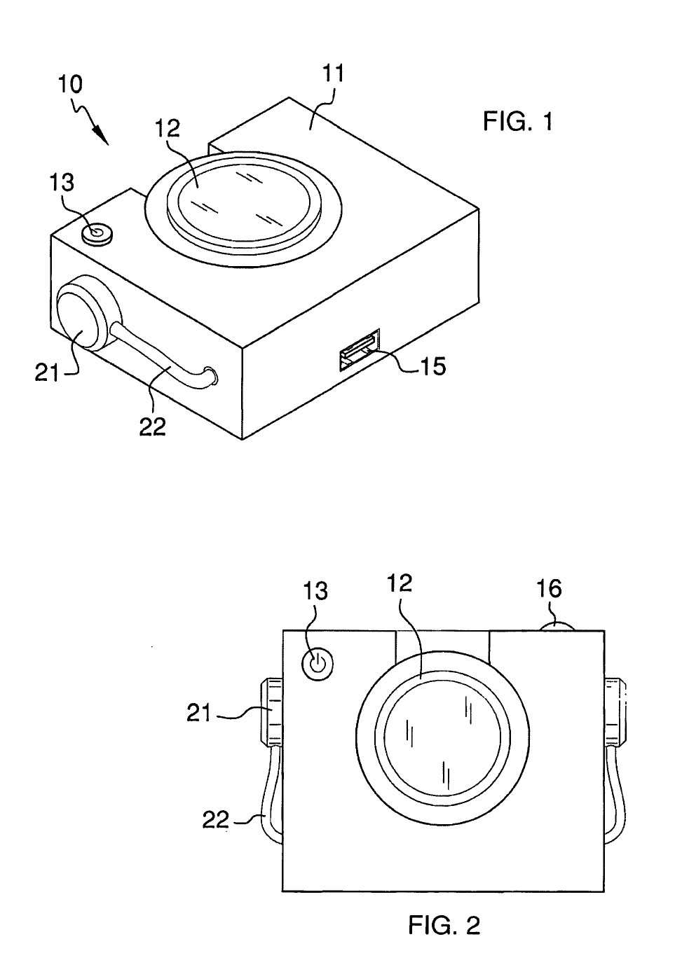

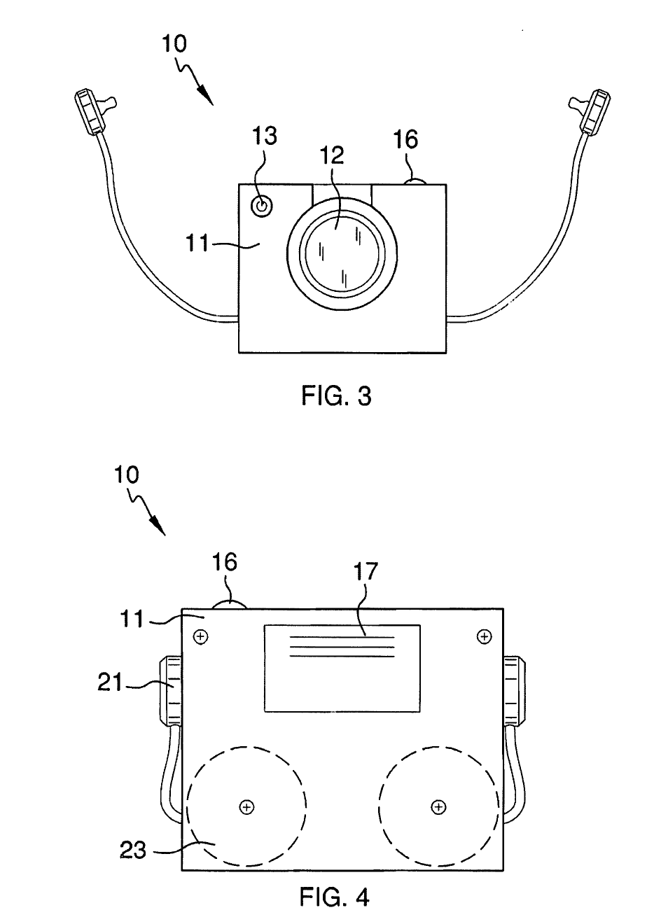

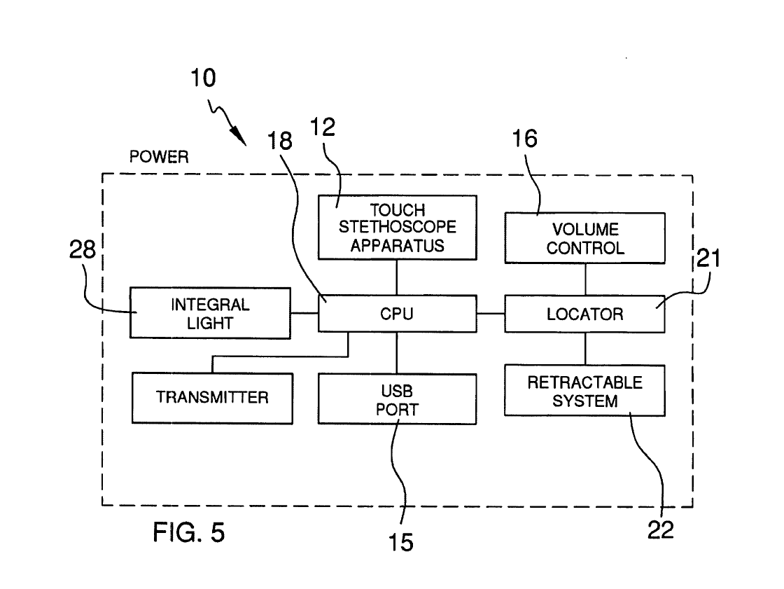

Detailed reference will now be made to the present invention, examples of which are illustrated in FIGS. 1-5. A first electronic stethoscope embodiment 10 (hereinafter invention) includes a housing 11, a stethoscope 12, an on/off switch 13, a retractable head phone set 14, an USB port 15, a volume control switch 16, a battery compartment 17, and a central processing unit (hereinafter CPU) 18.

The battery compartment 17 contains a plurality of rechargeable batteries (not shown) that supply electricity to the CPU 18. The CPU 18 is electrically wired to the stethoscope 12, and processes the sound from an analog signal to a digital signal. The CPU 18 is also wired to a memory stick (not shown) that is contained within the battery compartment 17. The memory stick records the digital sounds produced by the CPU 18. The CPU 18 is also wired to the retractable head phone set 14.

As depicted in FIG. 1, the invention 10 includes a USB port 15, which enables the end user to insert a USB memory stick (not shown) into the USB port 15 for extraction of audio files. The end user would then be able to insert the USB memory stick (not shown) into the USB port of a computer in order to transfer the audio file to a computer for further processing and/or review of the audio file.

The USB port 15 is wired to the CPU 18, which in turn retrieves the audio files from the memory stick (not shown) that is also wired to the CPU 18. The purpose of the memory stick (not shown) is to provide a temporary means of storing the audio files until extraction can be completed vis-à-vis the USB memory stick (also not shown).

The head phone set 14 includes a pair of head phones 21, a pair of head phone cords 22, and a pair of retractable systems 23, which unwinds and winds up the head phone cords 22. The retractable systems 23 are located within the housing 11.

Located along the exterior surface of the housing 11 is the ON/OFF switch 13 and the volume control switch 16, both of which are electrically wired to the CPU 18. The invention 10 also includes an integral light 28 for illuminating the various external componentry of the invention 10.

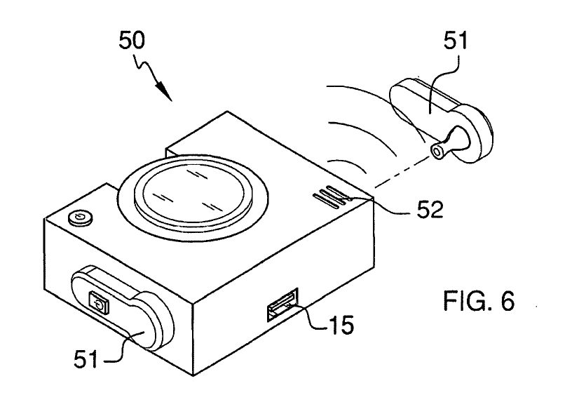

Referring to FIG. 6, an alternative embodiment 50 includes all of the above identified features of the first embodiment with the exception that the head phone set 14 are replaced with a pair of Bluetooth enabled head pieces 51, which receive a signal generated by a transmitter 52, which is electrically wired to the CPU 18 and transmits the digital signal produced by the CPU 18.

It shall be noted that an alternative embodiment (not depicted in the above referenced figures) shall include a small LCD display for the purpose of displaying an equalizer or sound-wave readout of the sounds generated by the patient's body.

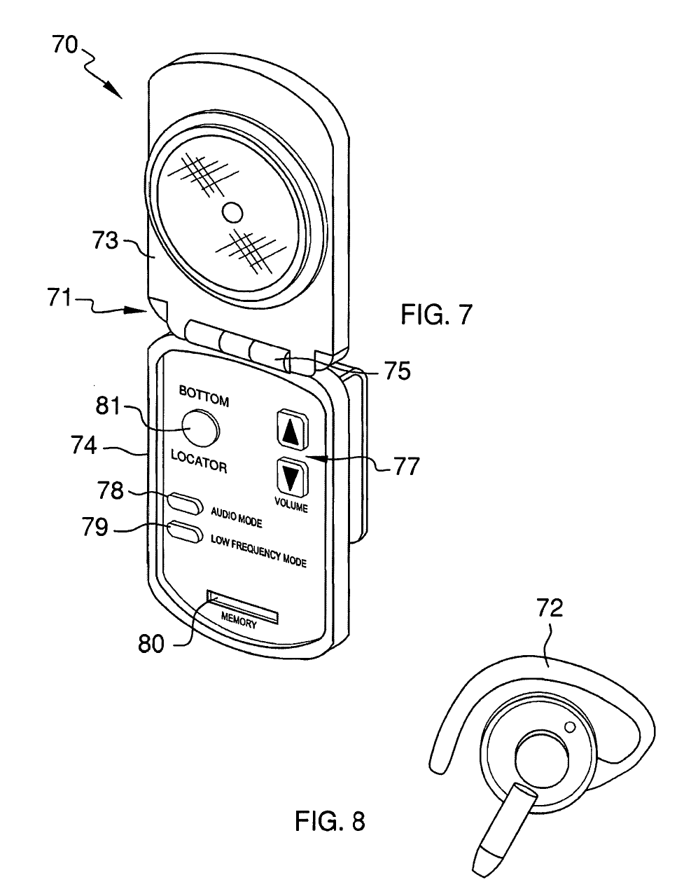

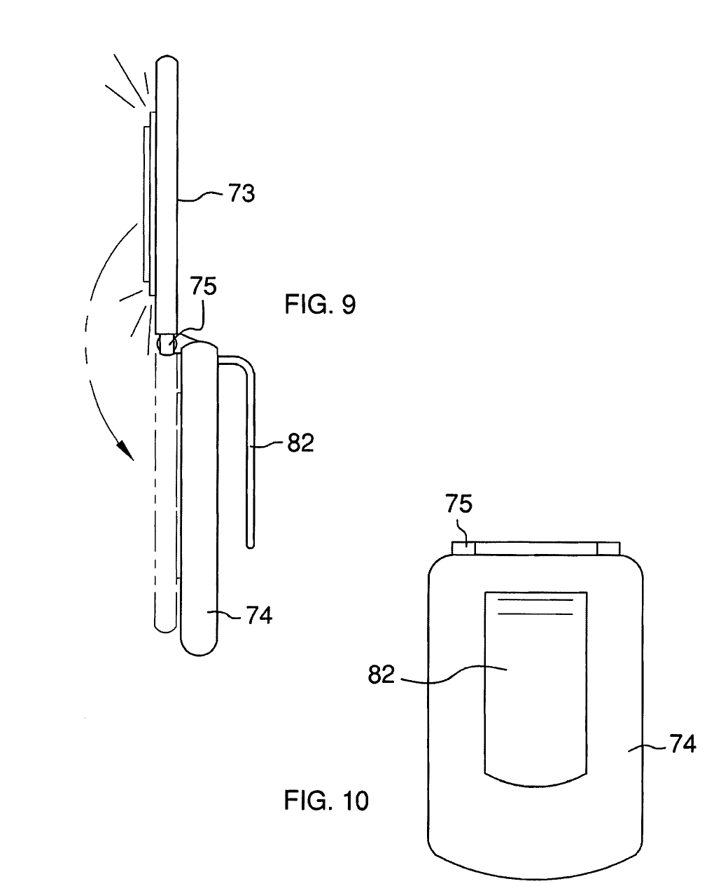

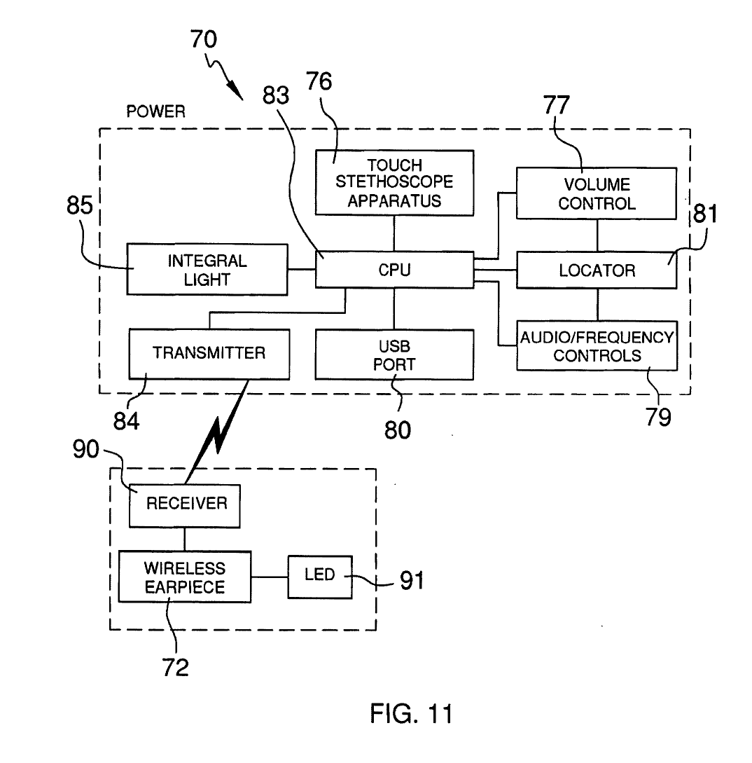

Referring to FIGS. 7-11, a flip-up embodiment 70 includes a flip-stethoscope 71 and a Bluetooth enabled head piece 72. The flip-stethoscope 71 features a top half 73 and a bottom half 74. The top half 73 is connected to the bottom half 74 by a hinge 75. A stethoscope 76 is located on the top half 73. Located on the bottom half 74 are volume buttons 77, an audio mode button 78, a low frequency mode button 79, a memory card slot 80, a bottom locator button 81, and a clip 82.

The flip-stethoscope 71 also includes a central processing unit 83 (hereinafter CPU), a transmitter 84, and an integral light 85.

The head piece 72 features a receiver 90, and an LED light 91. The LED light 91 is to indicate that the head piece 72 is operational. The transmitter 84 transmits a radio signal 92, which is received by the receiver 90.

It shall be noted that those skilled in the art will readily recognize numerous adaptations and modifications which can be made to the various embodiments of the present invention which will result in an improved invention, yet all of which will fall within the spirit and scope of the present invention as defined in the following claims. Accordingly, the invention is to be limited only by the scope of the following claims and their equivalents.

While the embodiments of the invention have been disclosed, certain modifications may be made by those skilled in the art to modify the invention without departing from the spirit of the invention.

The inventor claims:

1. A digital stethoscope and recording device comprising:
(a) a housing;
(b) a central processing unit (hereinafter CPU);
(c) an electronic stethoscope;
wherein the electronic stethoscope is wired to the CPU;
wherein the electronic stethoscope transmits the sounds from the stethoscope via an electrical signal to the CPU;
wherein the CPU processes the electrical signal into a digital signal;
(d) a USB port;
wherein the USB port is wired to the CPU;
wherein the USB port provides computer connectivity with the CPU;
wherein a USB memory stick may be inserted into the USB port for extraction of an audio file, and of which the USB memory stick can then transfer to a computer;
(e) a battery compartment;
wherein the battery compartment contains a plurality of rechargeable batteries;
wherein the rechargeable batteries provide electricity to the CPU;
(f) a memory stick;
wherein the memory stick is wired to the CPU;
wherein the digital sound processed by the CPU is sent to the memory stick where it is digitally recorded and stored;
wherein the USB memory stick extracts files that are stored with the memory stick via the CPU;
(g) an ON/OFF switch;
wherein the ON/OFF switch is wired to the CPU;
(h) a volume control;
wherein the volume control is wired to the CPU;
(i) a retractable head phone set;
wherein the retractable head phone set includes a pair of head phones, a pair of head phone cords, and a pair of retractable systems;
wherein the retractable systems are contained within the housing;
wherein the retractable housings wind and unwind the head phone cords; and
wherein the head phone cords are wired to the CPU.

2. The digital stethoscope and recording device as described in claim 1 wherein the retractable head phone set is replaced with a radio transmitter and a pair of wireless head phones, so as to provide the device a wireless capability.

3. The digital stethoscope and recording device as described in claim 1 wherein the housing contains a small LCD display that is electrically wired to the CPU;
wherein the LCD display provides a visual display of an audio signal that is created when in use; and
wherein the visual display of the audio signal is reflected as a sound-wave and/or an equalizer readout.

4. A digital stethoscope and recording device comprising;
(a) a top half;
wherein an electronic stethoscope is connected to the top half;
(b) a bottom half;
wherein the top half and the bottom half are connected by an attaching means;
(c) a head piece; and
wherein the head piece is in communication with the electronic stethoscope.

5. The digital stethoscope and recording device as described in claim 4 wherein the attaching means comprises a hinge.

6. The digital stethoscope and recording device as described in claim 4 wherein a memory card slot is provided that is capable of recording sounds produced by the electronic stethoscope.
